(19) United States
(12) Patent Application Publication
(10) Pub. No.: US 2004/0058311 A1
(43) Pub. Date: Mar. 25, 2004

(54) Method and apparatus for measuring the hemoglobin concentration and/or hematocrit in whole blood using diffuse light

(76) Inventors: Gary Fletcher, Wallingford, PA (US); Warren Groner, Great Neck, NY (US); Amy Perkins, Philadelphia, PA (US); Hsing-Wen Wang, Philadelphia, PA (US)

Correspondence Address: CAESAR, RIVISE, BERNSTEIN,, COHEN & POKOTILOW, LTD., 12TH FLOOR, SEVEN PENN CENTER, 1635 MARKET STREET, PHILADELPHIA, PA, 19103-2212, US

(21) Appl. No.: 10/362,644

(22) PCT Filed: Aug. 27, 2001
(86) PCT No.: PCT/US01/26652

(51) Int. Cl. C12Q 1/00

(52) U.S. Cl. 435/4

(57) ABSTRACT

A system, method and computer program product is provided for obtaining linear optical measurements (e.g., optical density) of hemoglobin concentration or hematrocrit in whole blood using diffuse illumination. The present invention uses a diffuse illumination source to measure spectral signatures. The light source is projected such that the optical measurement does not need to be corrected for scattering effects. The detected light in the present invention can be collimated light or light collected over a small solid angle and imaged onto a detector for accurate microvessel hematocrit measurements.

### BACKGROUND OF THE INVENTION

1. Field of the Invention

The present invention relates generally to reflected spectral imaging. More particularly, the present invention relates to correcting reflected spectral images for scattering effects to improve analysis of visualizable components within a fluid flowing in a tubular system.

2. Related Art

The development of techniques for microvessel hematocrit (Hct) determination using optical density (OD) measurement has posed a challenging problem since 1960s. The Lambert-Beer law of light absorption holds for hemoglobin in solution but not for whole blood due to light scattering from individual red blood cells. Further, the complex optical properties of whole blood lead to a nonlinear relationship between optical density and either the hemoglobin concentration or tube Hct. These phenomena have been studied and reported by several researchers. For example, wave-scattering phenomena is described in V. Twersky, “Multiple scattering of waves and optical phenomena,” J. Opt. Soc. Amer. 52, 145-171, 1962; V. Twersky, “Absorption and multiple scattering by biological suspensions,” J. Opt. Soc. Am. 60, 1084-1093, 1970; and V. Twersky, “Interface effects in multiple scattering by large, low-refracting, absorbing particles,” J. Opt. Soc. Am. 60, 908-914, 1970. In these references, Twersky proposes a wave-scattering theory that agrees reasonably well with experimental data.

This subject is also addressed in R. J. Jendrucko and J. S. Lee, “The measurement of hematocrit of blood flowing in glass capillaries by microphotometry,” Microvasc. Res. 6, 316-331, 1973; H. H. Lipowsky, S. Usami, S. Chien, and R. N. Pittman, “Hematocrit determination in small bore tubes from optical density measurements under white light illumination,” Microvasc. Res. 20, 51-70, 1980; J. M. Steinke and A. P. Shepherd, “Role of light scattering in spectrophotometric measurements of arteriovenous oxygen difference,” IEEE Trans. Biomed. Eng. BME 33, 729-734, 1986; and J. M. Steinke and A. P. Shepherd, “Role of light scattering in whole blood oximetry,” IEEE Trans. Biomed. Eng. BME 33, 294-301, 1986.

As an alternative, some researchers propose the use of photon-diffusion to provide a model of the optical transmittance of whole blood with some advantages. This theory is described in J. M. Schmitt, “Optical measurement of blood oxygen by implantable telemetry,” Ph.D. dissertation (Stanford University, Stanford, Calif., 1986); and J. M. Steinke and A. P. Shepherd, “Diffusion model of the optical absorbance of whole blood,” J. Opt. Soc. Am. A 5, 813-822, 1988.

In order to provide a practical measurement of hematocrit in-vivo, a differential wavelength method can be used to obtain a linear correlation of OD and Hct in whole blood. This method is discussed in H. H. Lipowsky, S. Usami, S. Chien, and R. N. Pituman, “Hematocrit determination in small bore tubes by differential spectrophotometry,” Microvasc. Res. 24, 42-55, 1982.

Others have proposed the use of a single parameter calibration method to slightly improve the rms error in hematocrit determination as compared to the differential wavelength methodology described by Lipowsky et al. This single parameter calibration method is described by A. R. Pries, G. Kanzow, and P Gaehtgens, “Microphotometric determination of hematocrit in small vessels,” Am. J. Physiol., 1983.

However, presently, there exists no conventional technique that provides a simple optical Hct prediction without using a second wavelength or a complex (non-linear) parameter correction procedure.

### SUMMARY OF THE INVENTION

The present invention is directed to a method and apparatus for obtaining linear optical measurements (e.g., optical density) of hemoglobin concentration or hematocrit in whole blood using diffuse illumination. The present method uses a diffuse illumination source to measure spectral signatures. The light source is projected such that the optical measurement does not need to be corrected for scattering effects. The detected light in the present invention can be collimated light or light collected over a small solid angle and imaged onto a detector for accurate microvessel hematocrit measurements.

### BRIEF DESCRIPTION OF THE FIGURES

The present invention is described with reference to the accompanying drawings. In the drawings, like reference numbers indicate identical or functionally similar elements. Additionally, the left-most digit(s) of a reference number identifies the drawing in which the reference number first appears.

FIG. 1 shows a collimated illumination optical schematic;

FIG. 2 shows a diffuse illumination optical schematic;

FIG. 3a shows Monte Carlo simulated images;

FIG. 3b shows intensity profiles produced FIG. 3a;

FIG. 4 shows a comparison of collimated and diffuse illumination; and

FIG. 5 shows a comparison of diffuse illumination and differential spectrophotometzy.

### DETAILED DESCRIPTION OF THE PREFERRED EMBODIMENTS

The present invention is directed to a method and apparatus for obtaining linear optical measurements (e.g., optical density) of hemoglobin concentration or hematocrit in whole blood using diffuse illumination. The method and apparatus of the present invention can be used on any medium having high scattering effects. When using diffuse light to illuminate a whole-blood-filled tube or vessel, the effects on absorption measurements due to red blood cell scattering are cancelled and compensated.

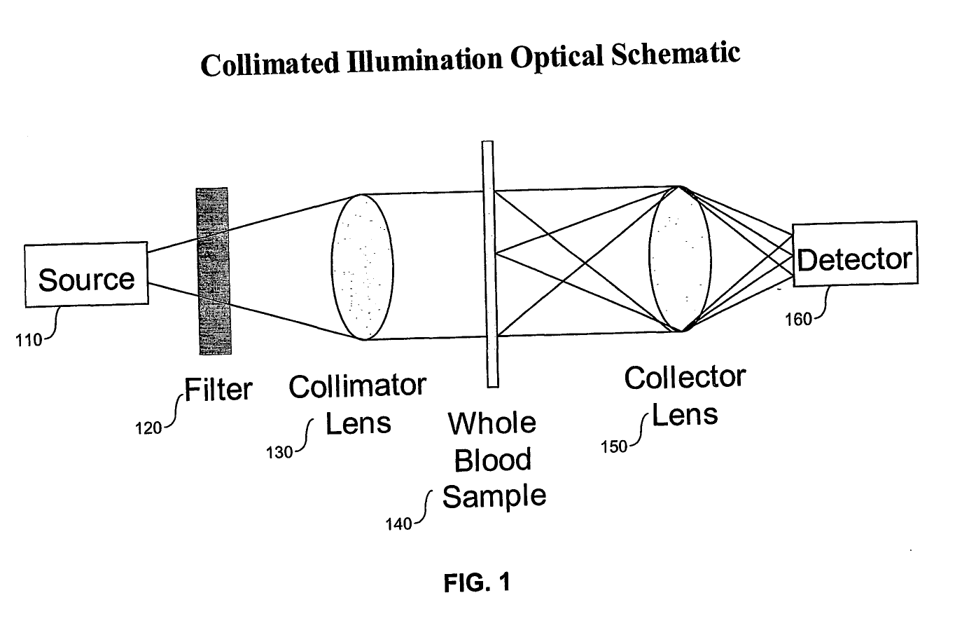

FIG. 1 illustrates a collimated illumination optical system. The system includes a source 110, filter 120, collimator lens 130, sample 140, collector lens 150 and detector 160. The system is part of a spectral imaging apparatus. The spectral imaging apparatus is preferably, but not necessarily, of the type described in commonly assigned U.S. Pat. No. 5,983,120, issued Nov. 9, 1999, in the names of Warren Groner and Richard G. Nadeau, and entitled “Method and Apparatus for Reflected Imaging Analysis” (hereinafter referred to as “the '120 patent”), or in commonly assigned U.S. Pat. No. 6,104,939, issued Aug. 15, 2000, in the names of Warren Groner and Richard G. Nadeau, and entitled “Method and Apparatus for Reflected Imaging Analysis” (hereinafter referred to as “the '939 patent”). The disclosures of the '120 patent and the '939 patent are incorporated herein by reference as though set forth in its entirety.

The device of the '120 patent or the '939 patent provides for complete non-invasive in vivo analysis of a vascular system. This device provides for high resolution visualization of blood cell components (red blood cells, white blood cells, and platelets), blood rheology, blood vessels, and vascularization throughout the vascular system. The device of the '120 patent or the '939 patent allows quantitative determinations to be made for blood cells, normal and abnormal contents of blood cells, as well as for normal and abnormal constituents of blood plasma. The device of the '120 patent or the '939 patent captures a raw reflected image of a blood sample, and normalizes the image with respect to the background to form a corrected reflected image.

It will be apparent to those skilled in the relevant arts that the spectral imaging apparatus contains many other components, including a housing, processing unit, etc. These elements are not shown for convenience of description of the inventive features.

Referring to FIG. 1, source 110 is a source of radiation (e.g., a light bulb) used to illuminate a region of interest. Filter 120 is a spectral selection means, such as a monochromator containing a prism, grating, colored filter or the like. Collimator lens 130 operates to focus and guide the illumination onto the region of interest. Sample 140 can be whole blood flowing in a vascular system. The spectral imaging apparatus supports imaging of sample 140 in vivo by imaging blood in a blood vessel or iii vitro by imaging blood in, for example, a tube or flow cell.

Collector lens 150 receives a reflected or transmitted image and projects the image to detector 160. Detector 160 can be photocells or the like that measures the amount of light reflected or transmitted by the sample 140 in the selected spectral region. As discussed, if sample 140 has high scattering effects (as is common for whole blood samples), the image received at detector 160 must be corrected.

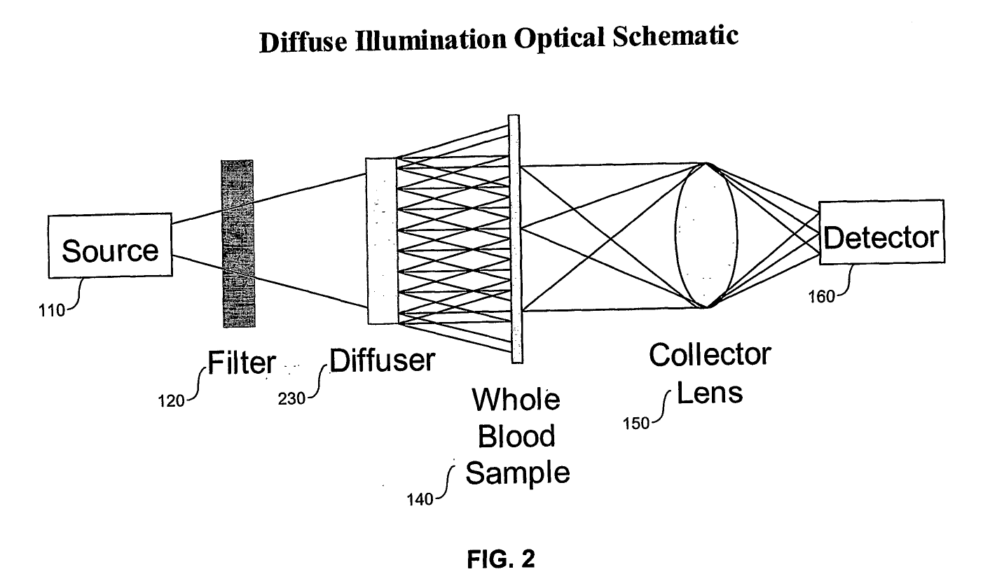

FIG. 2 illustrates an alternative illumination system that automatically corrects scattering produced in mediums with high scattering effects. FIG. 2 shows a diffuse illumination optical system for a spectral imaging apparatus, such as the device of the '120 patent or the '939 patent. As shown in FIG. 2, collimator lens 130 is replaced with diffuser 230. Diffuser 230 generates and projects a diffuse illumination pattern towards the region of interest. The illumination pattern is projected such that scattering is corrected. As a result, the image received at detector 160 would represent a true value of the transmitted or reflected image.

As described in greater detail below, a linear correlation of optical density (OD) and tube Hct using diffuse illumination can be demonstrated by implementing a Monte Carlo simulation. The results indicate that the method of the present invention, as shown in FIG. 2, can be used to obtain accurate microvessel Hct measurements. For comparison, results using collimated illumination (which shows the nonlinear correlation and was used in previous published studies) are illustrated below. Finally, a comparison is made between the diffused illumination concept and the differential wavelength method (which is commonly used today) in the range of interest.

### Monte Carlo Simulation Parameters

The geometrical setup of the systems studied is illustrated schematically in FIG. 1 and FIG. 2. A cylindrical capillary with 80 micrometer diameter is in air and illuminated by a 400×40-pixel area that is attached to the bottom of the capillary. The size of each pixel is 1 micrometer. At each pixel, N photons were sent through the system with single direction (collimated light, N=100) or random direction (diffuse light, N=200). To save program-running time, the angle of diffuse photon coming to the system was restricted with numerical aperture (NA) less than 0.436. The focus plane was set to the plane across the center of the capillary that is 40 micrometers above the illumination plane. The size of the image plane is 257×257 pixels. The pixel size is 1 micrometer. The NA of the objective collecting angle is 0.165.

Table 1 lists the optical properties for Hct=0.05 with illumination light at three wavelengths, namely at 500 nm, 550 nm, and 600 nm. (See, A. Roggan, M. Friebel, K. Dorschel, A. Hahn, and G. Muller, “Optical properties of circulating human blood in the wavelength range 400-2500 nm,” J. Biomedical Optics 4, 36-46, 1999.) Scattering and absorption coefficients for various Hct are linearly scaled by equations 1 and 2.

TABLE 1
Optical Properties

| | 500 nm | 550 nm | 600 nm |
|---|---|---|---|
| μ_{s.Hct5%}(1/cm) | 500 | 350 | 320 |
| μ_{a.Hct5%}(1/cm) | 15 | 30 | 5 |
| g | 0.99 | 0.99 | 0.99 |

$$\begin{matrix}{µ}_{a} = \frac{{µ}_{a , Hct5 \%} \times Hct}{0.05} & ( Eqn . 1 ) \\ {µ}_{s} = \frac{{µ}_{s , Hct5 \%} \times Hct \times ( 1 - Hct )}{0.005 \times ( 1 - 0.05 )} & \text{(Eqn. 2)}\end{matrix}$$

### Results

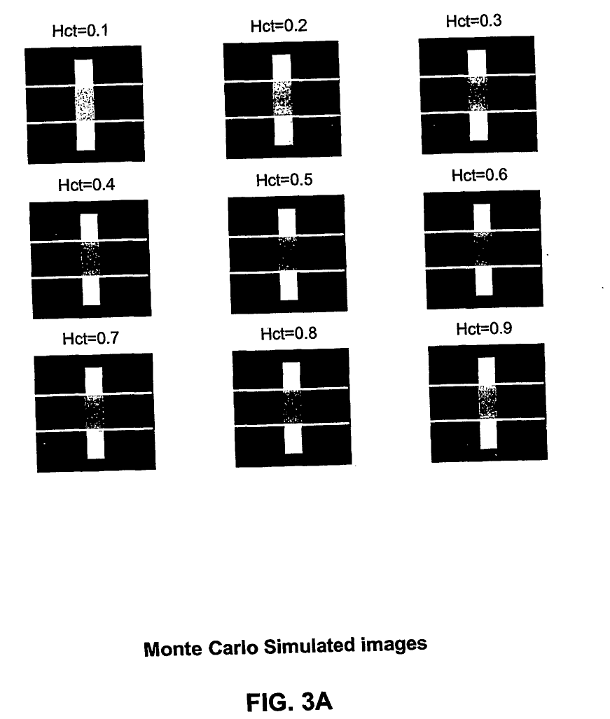

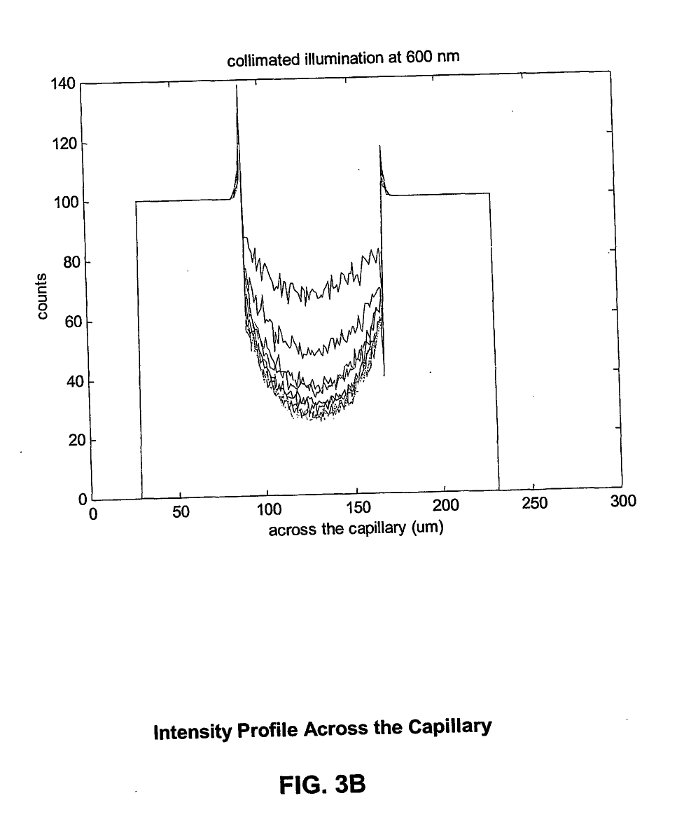

FIG. 3a and FIG. 3b show an example of capillary images (for Hct from 0.1 to 0.9), illuminated by collimated light, and their average intensity profile. Yellow lines outline the location of a capillary in each image.

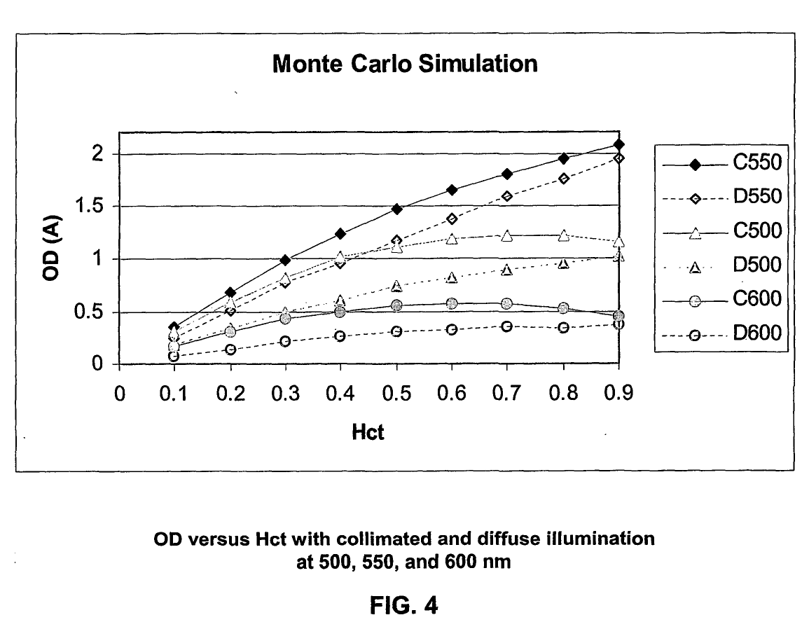

Optical density (OD) is defined as the logarithm of the ratio of background intensity (Ib) to vessel intensity (Iv). Ib is obtained by averaging a 10×10-pixel box at each side of the capillary with center shift by 70 pixels from the center of the capillary. Iv is obtained by averaging a 10×10-pixel box in the center of the capillary. A plot of OD versus Hct is shown in FIG. 4. In FIG. 4, the nonlinear correlation of OD and Hct using collimated illumination is shown at the three wavelengths, namely 500 nm, 550 nm and 600 nm. As shown in the legend to FIG. 4, “C” represents collimated illumination, and “D” represents diffuse illumination. As can be seen, diffuse illumination provides better response in terms of linearity, particularly at 550 nm.

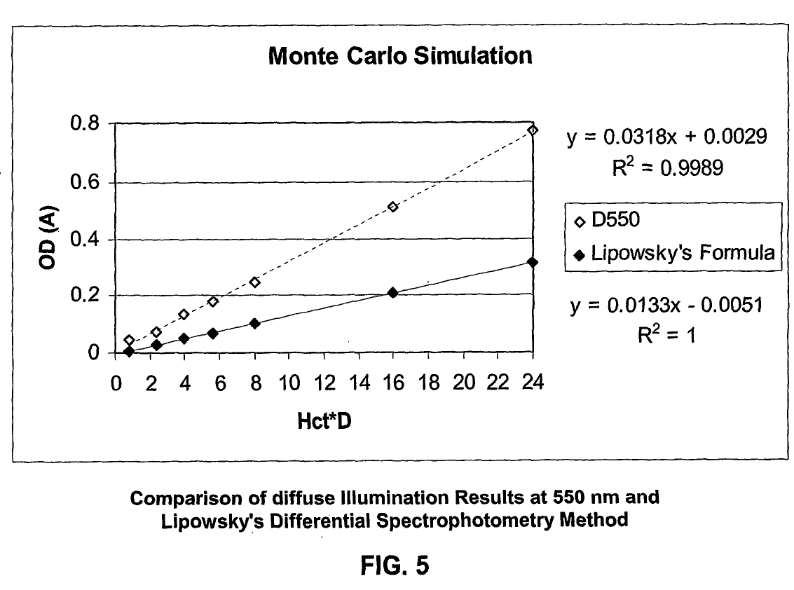

FIG. 5 shows a comparison between a diffuse illumination result at 550 nm and Lipowsky's differential method (two wavelengths) for the range of Hct*D (D=80 micrometers) from 0 to 24. As shown in FIG. 5, the intercept between these two methods is statistically equivalent (i.e., 0.9989 versus 1). Also, shown in FIG. 5, the sensitivity (i.e., slope) for the diffuse illumination results is at least two times higher than the sensitivity from Lipowsky's method (i.e., 0.0318 versus 0.0133).

While various embodiments of the present invention have been described above, it should be understood that they have been presented by way of example, and not limitation. It will be apparent to persons skilled in the relevant art(s) that various changes in form and detail can be made therein without departing from the spirit and scope of the invention. Thus, the present invention should not be limited by any of the above described exemplary embodiments.

What is claimed is:

1. A method for analyzing visualizable components in a fluid flowing in a vascular system, the walls of which are substantially transparent to transmitted and reflected light, using a light transmitting device that is capable of transmitting light through the vascular system, an image capturing device capable of capturing images from the vascular system illuminated by the light transmitting device to create a spectral image, and a processing unit in communication with the image capturing device, comprising the steps of:
transmitting light from the light transmitting device through a diffuser prior to transmitting the light through the vascular system, wherein said diffuser corrects scattering effects produced in the vascular system;
receiving in the processing unit a reflected spectral image of the vascular system captured by the image capturing device; and
analyzing said reflected spectral image to measure an optical density of the vascular region to calculate one of a hemoglobin concentration or hematocrit.

2. The method according to claim 1, wherein the fluid comprises blood flowing in a blood vessel of a mammalian vascular system, and the visualizable components comprise blood components, including red blood cells, white blood-cells, platelets, etc., and wherein said receiving step comprises receiving in the processing unit a reflected spectral image of the blood components in the region of the vascular system through which the light is transmitted and reflected.

3. An apparatus for analyzing visualizable components in a fluid flowing in a vascular system, the walls of which are substantially transparent to transmitted and reflected light, comprising:
a light transmitting device for transmitting light through the vascular system, wherein said light transmitting device includes a diffuser, said diffuser transmitting diffused light through the vascular system, wherein said diffused light corrects scattering effects produced in the vascular system;
an image capturing device for capturing images from the vascular system illuminated by said light transmitting device to create a spectral image; and
a processing unit adapted for communication with said image capturing device for receiving said spectral image, wherein said processing unit analyzes said spectral image to measure an optical density of the vascular region to calculate one of a hemoglobin concentration or hematocrit.

4. The apparatus of claim 3, wherein said light transmitting device further comprises:
a spectral selection means for filtering said light prior to transmitting said light through said diffuser.

5. The apparatus of claim 3, wherein said spectral selection means includes a colored filter.

6. The apparatus of claim 3, wherein said spectral selection means includes at least one of a prism and a grating.

7. The apparatus of claim 3, wherein said image capturing device further comprises:
a collector lens for receiving said spectral image.

8. The apparatus of claim 3, wherein said image capturing device further comprises:
a detector for measuring light reflected or transmitted by the vascular system.
